(19) United States
(12) Patent Application Publication
(10) Pub. No.: US 2013/0203830 A1
(43) Pub. Date: Aug. 8, 2013

(54) Compounds for Prevention of Cell Injury

(71) Applicants: Fatemeh Talaei, Groningen (NL); Robert Henk Henning, Groningen (NL); Adrianus C. Van der Graaf, Groningen (NL)

(72) Inventors: Fatemeh Talaei, Groningen (NL); Robert Henk Henning, Groningen (NL); Adrianus C. Van der Graaf, Groningen (NL)

(73) Assignee: SULFATEQ B.V., Groningen (NL)

(21) Appl. No.: 13/640,303

(22) PCT Filed: Apr. 18, 2011
(86) PCT No.: PCT/EP11/56146

§ 371 Date: Nov. 29, 2012

(30) Foreign Application Priority Data
Apr. 16, 2010 (NL) 2004569

(51) Int. Cl. C07D 311/04; C07D 233/64; C07C 211/27; C07C 39/06; C07D 209/14; C07C 211/29

(52) U.S. Cl. CPC C07D 311/04; C07D 209/14; C07C 211/29; C07C 211/27; C07C 39/06; C07D 233/64
USPC 514/400; 514/415; 548/504; 562/449; 514/567; 514/653; 564/443; 514/731; 568/781; 548/495; 514/419; 548/335.5; 562/444; 514/456; 549/405; 549/407; 435/1.1; 435/374

(57) ABSTRACT

The invention is related to compounds for prevention of cell injury or protection of cells. The compounds are involved in the maintenance or the increase of hydrogen sulphide in cells, which results in a protection of the cells or the prevention of cell injury. The compounds of the invention can be used in cell culture and tissue culture techniques. They can also be used in several medical conditions such as ischemia, reperfusion and hypothermia, or for preserving organs which are used for transplantation.

The present invention relates to a compound for use in the prevention of cell injury and/or protection of a cell against cell injury. The invention is also related to a compound that prevents cell injury and/or protects cells against injury in subjects suffering from a disorder that mediates oxidative stress to cells. Further, the invention relates to a pharmaceutical composition for use in diseases in human subjects wherein cell injury is involved, such as ischemia/reperfusion, inflammation, edema, hypothermia, stroke, hemorrhagic shock, diabetes. The invention is also related to the use of compounds for the protection of cells against injury, in vitro; for the protection of cells against injury in tissue culture ex vivo; and for the protection of cells in organs against injury e.g. during storage and preservation before transplantation. In addition, the invention relates to a method for the protection of cells or preventing cell injury in cells, tissues and organs.

Cell injury is caused by several factors such as oxygen deprivation (hypoxia/ischemia), extremes of temperature, certain chemical agents, infectious agents, immunological reactions, genetic defects and nutritional imbalances. Cell injury is caused when cells are stressed so severely that they are no longer able to adapt and the cell undergoes certain changes which leads to cell injury. The injured cells may recover from the injury (reversible stage) or the injury can culminate which leads to irreversible cell death.

The main mediators of cell injury are oxygen-derived free radicals (especially superoxide and hydroxyl radical) and high-energy oxidants (such as peroxynitrite).

The prevention of cell injury or protection of cells is required in several medical conditions such as ischemia/reperfusion, inflammation, hemorrhagic shock, diabetes and hypothermia. The prevention of cell injury or the protection of cells is also required to preserve organs which are e.g. used for transplantation, for therapeutic hypothermia.

Therapeutic hypothermia is used to protect biological material against injuries or degradative processes and is widely used in experimental and especially in clinical applications. Therapeutic hypothermia is a medical treatment that lowers a patient's body temperature to treat people having a condition or having the risk of obtaining a condition such as neonatal encephalopathy, cardiac arrest, ischemic stroke, traumatic brain injury, spinal cord injury, and neurogenic fever following brain trauma. It can be used to help reduce the risk of e.g. the ischemic injury to tissue during a period of insufficient blood flow. Periods of insufficient blood flow may be due to cardiac arrest or the occlusion of an artery by e.g. an embolism.

Although hypothermia has proven to have beneficial results, it is very often related to adverse effects such as arrhythmia, decreased clotting threshold, increased risk of infection, and increased risk of electrolyte imbalance. It has been proven that hypothermia is strongly injurious to a variety of cell types which may result in apoptosis, e.g. lung and heart cells. It is also found that there is a role of reactive oxygen species in hypothermic injury to these cells. Reactive oxygen species contribute to hypothermic injury in diverse mammalian cells such as liver and kidney cells. The hypothermic injury and the cold induced apoptosis occur upon rewarming of the cells after a period of cold incubation. Hypothermia and warming of the cells, is considered to have similar cell injury effects as when ischemia/reperfusion injury occurs.

Reperfusion injury refers to damage to tissue caused when blood supply returns to the tissue after a period of ischemia. The absence of oxygen and nutrients from blood during ischemia creates a condition in which the restoration of circulation (reperfusion) results in inflammation and oxidative damage through the induction of oxidative stress rather than restoration of normal function. Due to the oxidation there is an increase of free radical production which induces cells and tissue injury. The reintroduced oxygen also damages cellular proteins, DNA, and the plasma membrane. Damage to the cell's membrane may in turn cause the release of more free radicals. Such reactive species may also act indirectly in redox signaling to turn on apoptosis.

Organ transplantation is currently the preferred treatment option for patients suffering from end-stage failure of vital organs. After procurement from a donor, the immediate threat to organs is ischemia, which initiates complex injury processes. Therefore, ischemic injury is minimized by rapid in situ flushing with specific solution and cooling down the organs. Hypothermic storage of the organs at about 4° C. in a preservation solution, which primarily prevent injury by reducing ionic shifts during cold preservation, but do not affect apoptotic rate, is currently the main strategy in organ preservation before transplantation. However, continuation of cold ischemia and hypothermia-induced injury seriously damage organs.

Hydrogen sulphide (H₂S) is a newly found gasotransmitter which is endogenously produced by the enzymes cystathionine c-lyase (CSE) and cystathionine b-synthase (CBS). It has been suggested that H₂S has a cytoprotective effect against oxidative stress in cells. It has been shown that hydrogen sulfide (H₂S) plays key roles in a number of biological processes, including vasorelaxation, inflammation, apoptosis, ischemia/reperfusion and oxidative stress. It has also been shown that H₂S is protective against cardiac, hepatic, cerebral and renal ischemia/reperfusion injury. H₂S has also been shown to inhibit leukocyte-endothelial cell interactions in vivo, indicating an anti-inflammatory action. It strengthens cell barrier function and prevents cellular swelling. H₂S may thus function as an agent that protects cells against cell injury.

It has also been found that H₂S can induce suspended animation by decreasing the oxygen demand in cells. This confers protection against potentially lethal hypoxia. Suspended animation is a fascinating phenomenon consisting in lowering metabolic rate and increasing resistance to low oxygen concentration.

Further, it has been suggested that H₂S is a potential mediator of antioxidant and anti-apoptotic signalling which results in the induction of cell survival signalling pathway. It has been demonstrated that H₂S effectively inhibits apoptosis of a number of cell types. H₂S activates pathways that increase the level of glutathione and enhance the activity of KATP channels. Therefore it has been suggested that H₂S can protect cells from oxidative stress.

Most of these beneficial effects induced by H₂S were revealed in studies by applying exogenous donors of H₂S, such as sulfide salts (NaHS), which convert in H₂S. Since H₂S is as such a toxic gas for human beings, it is important to find other ways to upregulate the basal production of H₂S, i.e. the naturally occurring enzymatic release. It is thus important to find organic compounds that are able to release the gasotransmitter H₂S.

As described above, there are several factors that cause cell injury. Cell injury is mainly mediated by oxygen derived free radicals, intracellular increase of Ca²⁺, membrane damage and ATP depletion in the cell. If an injurious stimulus is severe or persists, the cell death can occur through necrosis or apoptosis. Ischemia is an example of a condition that induces cell injury through ATP depletion due to a decrease in oxidative phosphorylation. There are several agents, such as chemicals that induce disruption of the Ca²⁺ homeostasis. Cell injury due to reactive oxygen species and radicals are for example caused by inflammation, radiation, oxygen toxicity, chemicals, reperfusion injury. Diseases that induce cell injury due to oxidative stress are e.g. hemorrhagic shock; ischemic/reperfusion injury in heart, brain, liver, lungs, kidneys, and other tissues; inflammatory diseases, hypothermia, diabetes, thrombosis, edema. Consequently, there is a need for a compound which prevents cells against cell injury and protects cells in several diseases.

Cell injury also occurs in organs and tissues that are outside a subject, ex vivo and in cells for in vitro-use. Consequently, there is a need for a compound which prevents cells against cell injury and protects cells in organs and tissues ex vivo.

As described above, therapeutic hypothermia has been proven to be successful. However, side effects due to cell injury occur. There is a need for finding a compound which can protect the cells or prevent injury of the cells when performing therapeutic hypothermia.

As described above, suspended animation is an emerging method for treating subjects, and is induced by increase of the H₂S production in a cell. There is a need to provide compounds which can be used for inducing suspended animation.

It is in object of the present invention, among other objects, to provide a compound that helps preventing cell injury and/or protects cells against cell injury.

Since recent studies suggest that the gasotransmitter H₂S plays an important role in the induction of protection mechanisms in cells, it is another object of the present invention, amongst other objects, to provide a compound which induces endogenous production of H₂S in the cell.

It is yet another object of the invention to provide a compound that prevents cells or protects cells against injury, which occurs in several medical conditions such as hemorrhagic shock; ischemic/reperfusion injury in heart, brain, liver, lungs, kidneys, and other tissues; hypothermia, thrombosis, edema.

Further it is another object of the invention to provide a compound that prevents cell injury or protects cells against injury caused by inflammatory diseases and immunological reactions.

In addition, it is an object of the invention to provide a compound which prevents cell injury or protects cells against cell injury, used for cell culture and other cellular applications, against injury.

Further, it is an object of the invention to provide a compound which is used for protection of cells in tissues and organs, ex vivo.

In addition, it is an object of the invention to provide a compound which is used in the protection of cells against adverse effects that occur during hypothermic therapy.

It is yet another object of the invention to provide a compound, which can reduce the metabolism of a subject by increasing the H₂S production and/or inducing suspended animation.

The above objects, amongst other objects, are met at least partially, if not completely, by a compound as defined in the appended claim 1.

Especially, the above objects, amongst other objects, are met at least partially, if not completely, by a compound capable of increasing or maintaining the H₂S level in a cell for use in the prevention of cell injury and/or protection of cells.

The compound of the invention is capable of increasing or maintaining endogenous H₂S production in a cell for use in the prevention of cell injury and/or protection of a cell.

The compound of the invention is capable of increasing or maintaining endogenous H₂S production in a cell for use in the prevention of cell injury and/or protection of a cell, wherein the compound is selected from the group consisting of serotonin, baclofen, dopamine, propofol, melatonin, histamine, D/L phenylserine, trolox, reduced trolox and/or a salt, a derivate, or a precursor thereof.

The inventors have surprisingly found that the compound according to the invention increases the H₂S production in a cell and increases the H₂S level in a cell.

According to this invention, the increase of the H₂S level in the cell is an increase of the H₂S concentration in the cell as a total, or at a local place in the cell, which is higher than what is usually found in the cells or at that local place of the cell when none of the compounds of the invention are used, but under the same conditions. The production of H₂S in cells increases 1.5 to more than 20-fold after providing the compound according to the invention, compared to the baseline production in non-stimulated cells. The production of H₂S increases e.g. about 1.5, 2, 3, 4, 5, 6, 7, 8, 9, 10, 15, 20, or 25 fold.

H₂S is a gas and can easily diffuse through the cellular membrane. The increase of the H₂S production in the cell can lead to an increase of H₂S concentration outside the cell after the gas diffuses through the membrane. H₂S outside the cell can again diffuse back inside the cell. H₂S can function as preconditioning agent both inside and outside the cell and can protect cells against injury by e.g. inhibiting the progression of apoptosis, increasing the expression, upregulating or activating several proteins that mediate cell protection against injury (e.g. HSP-90, HSP-70, Bcl-Xl and Bcl-2). It can also inhibit cell injury by activation pathways that are involved in cell protection (e.g. the Akt pathway) and regulate gene transcription of genes involved in cell injury, or in protection against cell injury.

Accordingly, the compound of the invention mediates the increase of the H₂S production in a cell that functions as a preconditioning agent or a cytoprotective molecule. The compound of the invention brings the cell back in a normal state or keeps the cell in a normal state and prevents that cell injury occurs which can lead to apoptosis or necrosis. The compound of the invention can mediate as an anti-apoptotic signalling molecule or an antioxidant.

The compound of the invention is selected from the group consisting of serotonin, baclofen, dopamine, propofol, melatonin, histamine, D/L phenylserine, trolox and reduced trolox. These compounds are known, and have the following structural formula.

### Serotonin:

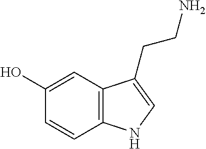 [SMILES: NCCc1c[nH]c2ccc(O)cc12]

Baclofen or (RS)-4-amino-3-(4-chlorophenyl)butanoic acid

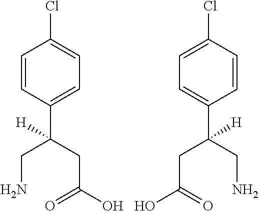

Dopamine or 2-(3,4-dihydroxyfenyl)ethylamine

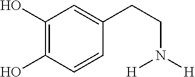 [SMILES: NCCc1ccc(O)c(O)c1]

Propofol or 2,6-diisopropylfenol

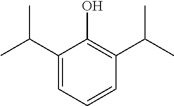

Melatonin or N-[2-(5-methoxy-1H-indol-3-yl)ethyl]acetamide

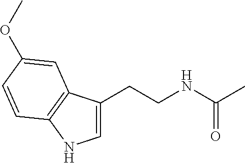 [SMILES: COc1ccc2[nH]cc(CCNC(C)=O)c2c1]

Histamine or 4-(2′-aminoethyl)-imidazol

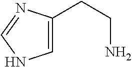

D/L phenylserine

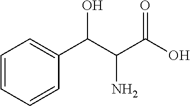 [SMILES: NC(C(=O)O)C(O)c1ccccc1]

### Trolox

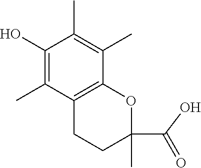 [SMILES: Cc1c(C)c2c(c(C)c1O)CCC(C)(C(=O)O)O2]

Reduced trolox,

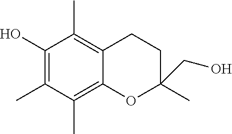 [SMILES: Cc1c(C)c2c(c(C)c1O)CCC(C)(CO)O2]

In one embodiment, the compounds of the invention are a derivate, salt or precursor of serotonin, baclofen, dopamine, propofol, melatonin, histamine, D/L phenylserine, trolox, or reduced trolox.

In one embodiment of the invention serotonin is capable of increasing or maintaining endogenous H₂S production in a cell for use in the prevention of cell injury and/or protection of a cell.

In one embodiment of the invention baclofen is capable of increasing or maintaining endogenous H₂S production in a cell for use in the prevention of cell injury and/or protection of a cell.

In one embodiment of the invention dopamine is capable of increasing or maintaining endogenous H₂S production in a cell for use in the prevention of cell injury and/or protection of a cell.

In one embodiment of the invention propofol is capable of increasing or maintaining endogenous H₂S production in a cell for use in the prevention of cell injury and/or protection of a cell.

In one embodiment of the invention melatonin is capable of increasing or maintaining endogenous H₂S production in a cell for use in the prevention of cell injury and/or protection of a cell.

In one embodiment of the invention histamine is capable of increasing or maintaining endogenous H₂S production in a cell for use in the prevention of cell injury and/or protection of a cell.

In one embodiment of the invention D/L phenylserine is capable of increasing or maintaining endogenous H₂S production in a cell for use in the prevention of cell injury and/or protection of a cell.

In one embodiment of the invention trolox is capable of increasing or maintaining endogenous H₂S production in a cell for use in the prevention of cell injury and/or protection of a cell.

In one embodiment of the invention reduced trolox is capable of increasing or maintaining endogenous H₂S production in a cell for use in the prevention of cell injury and/or protection of a cell.

In one embodiment of the invention, a combination of two or more compounds selected from the group consisting of serotonin baclofen, dopamine, propofol melatonin, histamine, D/L phenylserine, trolox, and/or a salt, a derivate, or precursor thereof is used for increasing the H₂S production in the cell.

In one embodiment of the invention, a combination of dopamine and propofol or a salt, a derivate, or precursor thereof is used for increasing the H₂S production in the cell.

According to this invention, cell injury is defined as an alteration in cell structure or functioning resulting from stress that exceeds the ability of the cell to compensate through normal physiologic adaptive mechanisms.

It is surprisingly found that the compound of the invention protects cells form cell injury which is caused by several conditions.

In one aspect, this invention is related to the prevention of cell injury or the protection of the cell against cell injury, caused by the factors: oxygen deprivation (hypoxia and ischemia); physical agents (such as mechanical trauma, extremes of temperature, burns and deep cold, sudden changes in atmospheric pressure, radiations, electric shock); chemical agents and drugs; infectious agents; immunologic reactions; genetic diseases; or nutritional imbalances.

In one embodiment, the protection against cell injury is provided by a compound according to the invention selected from the group consisting of serotonin, baclofen, dopamine, propofol, melatonin, histamine, D/L phenylserine, trolox, reduced trolox and/or a salt, a derivate, or a precursor thereof, wherein the compound induces anti-apoptotic signalling mediated by the increases of H₂S production in a cell.

In another embodiment, the protection against cell injury is provided by a compound according to the invention selected from the group consisting of serotonin, baclofen, dopamine, propofol, melatonin, histamine, D/L phenylserine, trolox, reduced trolox and/or a salt, a derivate, or a precursor thereof, wherein the compound protects the cell against injury caused by oxygen deprivation (hypoxia and ischemia); physical agents (such as mechanical trauma, extremes of temperature, burns and deep cold, sudden changes in atmospheric pressure, radiations, electric shock); chemical agents and drugs; infectious agents; immunologic reactions; genetic diseases; or nutritional imbalances. The invention is related to the use of the compounds in treatment of subjects suffering from cell injury caused by oxygen deprivation (hypoxia and ischemia); physical agents (such as mechanical trauma, extremes of temperature, burns and deep cold, sudden changes in atmospheric pressure, radiations, electric shock); chemical agents and drugs; infectious agents; immunologic reactions; genetic diseases; or nutritional imbalances.

According to this invention, hypoxia means depriving cells, tissues or organs of oxygen. Hypoxia can result from interrupted blood supply (ischemia), inadequate oxygenation of blood due to pulmonary disease or hypoventilation, inability of the heart to adequately pump blood (heart failure), or impaired oxygen carrying capacity of the blood (anemia, carbon monoxide poisoning, etc.). Hypoxia depletes cellular ATP and generates oxygen-derived free radicals.

According to this invention chemical injury can be caused by a very large number of drugs and environmental chemical agents that are capable of causing cell injury, including inorganic compounds, ions, and organic molecules—including byproducts of normal metabolism and toxins synthesized by microorganisms. The mechanism of chemical injury to cells ultimately rely on the activation of common injury pathways in cells, including e.g. interference with the function of critical molecules, either directly or via the production of toxic compounds, including oxygen radicals.

According to this invention physical agents can be harmful to cells and tissues. Common examples include: mechanical injury (crush injury, fractures, lacerations, hemorrhage), extremes of heat or cold (burns, heat stroke, heat exhaustion, frostbite, hypothermia), ionizing or non-ionizing radiation—(x-rays, radioactive elements, ultraviolet radiation), electric shock, sudden changes in atmospheric pressure (blast injury, decompression injury in divers), noise trauma. These ultimately activate cell death programs, either through direct loss of cell integrity, or through activate of various intracellular messenger pathways.

According to this invention, cell injury caused by infection results from the colonization of the body by pathogenic viruses, bacteria, fungi, protozoa, or helminths. Pathogenic organisms produce disease by either: (1) replicating inside host cells and disrupting the structural integrity of the cell (direct cytopathic effect), (2) producing a toxin that is harmful to host cells, or by (3) triggering an inflammatory or immune response that inadvertently injures host cells caught in the “cross fire” between the immune system and invading microorganism.

According to this invention, cell injury can be caused by immune reactions, which also include exaggerated immune reactions (anaphylaxis, allergy), or the inappropriate targeting of the body's own cells by the immune system (autoimmunity).

According to this invention, cell injury caused by nutritional imbalance means cell injury caused by a deficiency or an excess in normal cellular substrates.

According to this invention, cell injury caused by genetic derangements are the genetic derangements that are inherited or acquired mutations in important genes that can alter the synthesis of crucial cellular proteins leading to developmental defects, or abnormal metabolic functioning.

In another aspect of the invention, the prevention of cell injury and/or the protection of a cell against injury is achieved in the treatment of subjects suffering from a disorder that mediates oxidative stress to cells. The invention is related to the use of the compound of the invention in the treatment of subjects suffering from a disorder that mediates oxidative stress to cells. Oxidative stress represents an imbalance between the production and manifestation of reactive oxygen species and a biological system's ability to readily detoxify the reactive intermediates or to repair the resulting damage in a cell. Disturbances in the normal redox state of tissues can cause toxic effects through the production of peroxides and free radicals that damage all components of the cell, including proteins, lipids, and DNA. Disorders that mediate oxidative stress include but are not limited to hemorrhagic shock; ischemic/reperfusion injury in heart, brain, liver, lungs, kidneys, and other tissues; inflammatory diseases, hypothermia, thrombosis, edema, neuromodulation, hypertension, inflammation, diabetes, and hemorrhagic shock.

In another aspect, the invention is related to the use of the compound of the invention in the treatment of subjects suffering from hemorrhagic shock; ischemic/reperfusion injury in heart, brain, liver, lungs, kidneys, and other tissues; inflammatory diseases, hypothermia, thrombosis, edema, neuromodulation, hypertension, inflammation, diabetes, and hemorrhagic shock.

The inventors surprisingly found that the compound of the invention increases the activity of cystathionine beta synthase (CBS) in the cell. In yet another aspect, the compound of the invention induces CBS activity. CBS is an enzyme that converts homocysteine to cystathionine and produces H₂S. Cystothionine is then converted to L-Cysteine by cystathionine gamma lyase (CSE). L-cystathionine is then further converted by CSE or CBS, further producing H₂S.

In one embodiment, the increase of endogenous H₂S production is mediated by upregulating CBS. This means that the compound of the invention induces the increase of the amount or concentration of CBS in a cell.

In yet another embodiment, the increase of endogenous H₂S is mediated by activating CBS. The compound of the invention activates CBS for example through allosteric binding of the compound of the invention to the enzyme CBS, or by activating another compound which binds allosterically to the enzyme.

In yet another aspect of the invention, the compounds of the invention can be taken up in the cell via a transporter or via other means of active transport. For example serotonin is taken up by the serotonin transporter.

In one embodiment of the invention, the compound is used to prevent cell injury or to protect cells during the treatment of therapeutic hypothermia. The compound is used to limit adverse effects of therapeutic hypothermia. By the addition of the compound of the invention, during hypothermic therapy, the compound can limit cell injury by maintaining or increasing the H₂S level.

It is known that administration of H₂S to mice induces a reversible reduction in metabolism described as suspended animation. Suspended animation is the slowing of life processes by external means without termination. Breathing, heartbeat, and other involuntary functions are considerably, but reversibly, inhibited during this state.

It is known that an increase of H₂S production induces the reversible reduction in metabolism. Another aspect of the invention is the compound according to the invention for inducing suspended animation, wherein the increase of H₂S mediates or induces the suspended animation by adding the compound to the subject. In addition, the compounds can protect the cells and prevent cell injury by increasing the H₂S production in the cell during suspended animation.

In yet another aspect, the compounds of the invention are used to protect cells from organs, ex vivo, against injury. This is e.g. important during hypothermic storage of organs. During hypothermic storage and especially in the process of bringing the organs back to body temperature, severe damage to the organs occurs due to cell injury mainly caused by oxidative stress. The compound of the invention protects the cell against injury of the organs by increasing H₂S production in the cells. The compound can be administered to the organ donor before surgery. Another possibility is to add the compound to the preservation solution of the organ. The organs are protected against cell injury by adding the compounds of the invention during storage, cold storage (e.g. at temperatures between 0-25° C., which is about 0, 1, 2, 3, 4, 5, 6, 7, 8, 9, 10, 11, 12, 13, 14, 15, 16, 17, 18, 19, 20, 21, 22, 23, 24 or 25° C.) and warming up of the organ.

In another embodiment the compounds can be used to protect cells that are used in vitro and thus outside the body. When cells are stored, they are cooled down. Often the cells are frozen in liquid nitrogen. Rewarming or thawing of the cells for new use results in a great loss of cells and it often takes a long time before the cells start dividing again. The compound can be added to the solution that is used to freeze the cells so that cells are more protected from cell injury and it will be easier and faster to start a new cell culture. In addition the compound of the invention protects the cell, when it is added to the cells in a storing buffer, and stored at cold temperatures, e.g. between 0-8° C. preferably 4° C. In addition, the compound can be added to the growing medium of the cells, to provide further protection against injury, or to perform cellular experiments on the induction of H₂S.

Another embodiment is the use of the compound of the invention to protect cells from tissue cultures against cell injury ex vivo. Tissue cultures are preparations of unisolated cells maintained within its original architecture. The tissues are protected against cell injury by adding the compounds of the invention during storage, cold storage (e.g. at temperatures between 0-25° C., which is about 0, 1, 2, 3, 4, 5, 6, 7, 8, 9, 10, 11, 12, 13, 14, 15, 16, 17, 18, 19, 20, 21, 22, 23, 24 or 25° C.) and warming up of the tissue.

Another aspect of the invention is to prevent cell injury caused by conditions that induce cell injury, as described above, in subjects, wherein the compound of the invention is administered as a pharmaceutical composition which also comprises a pharmaceutically acceptable excipient.

For clinical use, the compounds of the invention are formulated into pharmaceutical formulations for oral, intravenous, subcutaneous, tracheal, bronchial, intranasal, pulmonary, transdermal, buccal, rectal, parenteral or other mode of administration. The pharmaceutical formulation contains a compound of the invention in combination with one or more pharmaceutically acceptable ingredients. The carrier may be in the form of a solid, semi-solid or liquid diluent, or a capsule.

In one embodiment, the pharmaceutical composition is locally administered via means such as a stent or a catheter.

In another aspect, the invention is related to a method for the protection of cells or preventing cell injury, comprising the addition of the compound of the invention or a composition comprising the compound of the invention, wherein the compound or the composition is added to cells before cooling the cells or wherein the compound or the composition is added to cells before warming the cells.

In another aspect, the invention is related to a method for preventing cell injury or the protection of cells in an organ or a tissue comprising the addition of a compound according to the invention or a composition comprising the compound of the invention, wherein the compound or the composition is added to the organ or the tissue before cooling the organ or the tissue, or before warming the organ or the tissue for prevention of cell injury and/or protection of cells.

The present invention will be further illustrated in the examples that follow. The examples are in no way intended to be limiting to this invention. In this description and the examples reference is made to the following figures.

### FIGURES

FIG. 1: A) Resistance of DDT-1 cells to hypothermia (24 h, 3° C.), B) Medium of hypothermia exposed DDT-1 cells (CM3; 18 h, 3° C.) protects vulnerable cell lines from hypothermia induced cell death (24 h, 3° C.) compared to unconditioned medium from DDT1 cells (CM37; 18 h, 37° C.). Data are the Mean±SEM (n=8). *<0.0001; unpaired t-test

FIG. 2: Different stainings of DDT-1 cells showing cytoplasmic expression of granules, which are decreased in number and intensity by hypothermia (3° C., 18 hrs); A) Typical example of live DDT-1 cells stained with methylene blue, B) Typical example of DDT-1 cells fixed by acetone and stained by Ehrlich reagent, C and D quantification of vesicle area (% of cytoplasmic area) and intensity (morphometry). E) Immunohistological staining of DDT-1 cells using serotonin specific antibody Data are the Mean±SEM (n=8). * p<0.005; ANOVA.

FIG. 3: Indoleamine concentration in hypothermic DDT-1 and SMAC cells. A) Indoleamine concentration in DDT-1 cells in hypothermic cells (3° C.) at different time points, B) Indoleamine concentration in hypothermic SMAC cells (3° C.) at different time points of cells pretreated with DDT-1 supernatant. Data are Mean±SEM (n=4) * p<0.005; ANOVA.

FIG. 4: DDT-1 cell survival following hypothermia is blocked by the tryptophan synthetase inhibitor parachlorophenylalanine (PCPA). Data are the Mean±SEM (n=8). * p<0.005 compared to control; ANOVA.

FIG. 5: Protection of hypothermic cell death by serotonin. A-C) SMAC cells 24 hrs after treatment by 3 different concentration of 5-HT at 3° C. (0.76, 1.17, 1.36 μM) D) % Area, Density/intensity of SMAC cells covering the bottom of the well at 3° C. applying 3 different concentrations of 5-HT E) Graph demonstrating the survival of SMAC cells after 24 hours of hypothermia treatment with different concentrations of 5-HT in supernatant, F) Caspase 3/7 assay for SMAC cells treated by 5-HT and DDT-1 HTM compared to controls treated with SMAC medium, G) The observed effect of Fluoxetin (0.001-1 μM) combined with Serotonin (13 nM) added to SMAC cells during hypothermic treatment compared to free serotonin and controls and the effect of Fluoxetin (0.005 μM) on DDT-1 survival at 3° C.

FIG. 6: Change in H₂S concentration in DDT-1 and SMAC cells and medium during 24 hr of hypothermic treatment compared to the control A) hypothermic DDT-1 and SMAC cells B) H₂S concentration in control and hypothermic DDT-1 and SMAC cell free medium. Data are the Mean±SEM (n=3. * p<0.005; ANOVA).

FIG. 7: Cystathionine-β-synthase (CBS) mediates protective effects against hypothermic cell death; A) DDT-1 cells stained with CBS antibody compared to control, B) SMAC cells stained with CBS antibody compared to control, C) siRNA against CBS decreases cell survival of DDT-1 cells during hypothermia compared to control and mock transfected cells D) siRNA against CBS annihilates the protective effect of serotonin on hypothermic cell death in SMAC cells. E) CBS expression after the addition of CBS siRNA to the DDT-1 at 37° C. F) CBS expression after the addition of CBS siRNA to the SMAC and the SMAC at 37° C. The Data are the Mean±SEM (n=8). * p<0.005; ANOVA.

FIG. 8: Serotonin treatment upregulates CBS expression in 37° C. SMAC cells. Confluent SMAC cells were incubated for the indicated min with serotonin (1.3 μM).

FIG. 9: Serum Indoleamine concentration in hibernating animals

FIG. 10: Upregulation of CBS in lung during the torpor phase in hibernating hamster. A1) CBS expression normalized to GAPDH at different phases of hibernation, A2) expression of CBS in Hamster blood pellet (average of 3 animals), B) EU, C) TE, D) TL E) EA

FIG. 11: Protein-ligand docking studies demonstrating the binding. C; Green, N; Blue, 0; oxygen, H; Hydrogen

FIG. 12: CBS enzyme (100 μg/ml) activity; substrates (10 mmol) and PLP (0.1 mM) added to the enzyme as control and serotonin (10 mmol) A) 37° C., B) 3° C.

FIG. 13: CBS expression in rat tissues, showing decreased expression in hypothermia (middle panels) and increased expression of hypothermic tissue treated with dopamine (right panels) A; liver, B; Pancreas, C; Lung, D; kidney, E; Heart.

37° C. (left panels: A1-E1), Control CBS expression in tissue 3° C. after 24 h (middle panels: A2-E2), CBS expression in Dopamine treated tissue 3° C. after 24 h (right panels: A3-E3)

FIG. 14: A: H₂S production in untreated SMAC cells (left columns) and treated with different compounds (right columns) at 24 h of hypothermic treatment at 3° C. B: H₂S production in treated SMAC cells with different compounds (25=D/L phenyl serine; 17=Reduced trolox; 11=trolox) of hypothermic treatment at 3° C., wherein the relative increase of the H₂S production is measured expressed as fold change over H₂S production in untreated SMAC cells at 3° C. (baseline).

FIG. 15: Caspace 3/7 activity in control and protective factor treated tissues, showing high expression of caspace for control groups and lower expression for dopamine treated group at 3° C. compared to 37° C. controls A; liver, B; Pancreas, C; Lung, D; kidney, E; Heart.

FIG. 16: Propofol protects liver tissue against in vivo hypothermia and induces expression of cystathionine beta synthase. Propofol treated samples were compared to control (gray bars, 37° C.) and hypothermic ketamine treated rat livers (black bars, 3° C.) after colling and rewarming. (A) fatty acid binding protein immunostaining in liver (40×) (B) CBS immunostaining in liver expression increased in the liver. (C) FABP expression obtained by western blot analysis in liver. (D) CBS expression by western blotting in liver tissue. ANOVA tests, different from non-cooled tissue (Controls 37° C.) P<0.05 (*); different from hypothermic tissue (ketamin 3° C.) P<0.05 (#). Experiments consist of n 3. Means±SEM.

FIG. 17: Serum H₂S concentration in rats throughout the procedure. H₂S concentration was compared to control (gray bars, 37° C.) for propofol (left bars, marked P) and ketamine (right bars marked K). X axis denotes body temperature of rats at sampling and anesthetic regime used. Experiments consist of n 3. Means±SEM.

### EXAMPLES

### 1. Material and Methods

### 1.1 Cell Culture and Hypothermic Insult

Cell lines used included NRK (normal rat kidney cells), DDT-1 (hamster ductus deferens muscle cells) and A7R5 (rat vascular smooth muscle cells) cultured in DMEM (Gibco, 41966, UK) and SMAC (rat smooth muscle aortic cells) and THMC (transformed human mesangial cell) cultured in DMEM/F12 (Gibco, E12-719F). All media were supplied with 10% (vol/vol) fetal calf serum and 1% penicillin-streptomycin and cultured at 37° C. in 5% CO₂ in 25 cm² or 75 cm² flasks. For hypothermia experiments, cells were plated in 6 or 96 wells plates and grown to confluence. Thereupon, cells were placed at 3° C. for up to 24 hours. Cell survival was measured by counting of trypan blue stained cells or MTS assay, which measures the number of viable cells. For the latter, 20 μl of MTS solution was added to each well and cells were subsequently placed in the incubator at 37° C. in 5% CO₂ for 3 hr before assessing the cell survival by measuring absorption using a microplate reader at 490 nm.

### 1.2 Conditioned Medium

DDT-1 cells were grown to confluence in 25 cm² flasks, washed with PBS (phosphate buffered saline), covered with 5 ml of medium and placed at 3° C. or 37° C. for 18 h to obtain conditioned medium (CM3 and CM37, respectively). CM was filtered through a 0.2 μm cellulose acetate disposable filter unit (Whatman, 0.2 μm cellulose acetate, 104962200) and stored at −20° C. until use. NRK, SMAC, A7R5 and THMC were grown to confluence in 96 well plates. Thereupon, the supernatant was replaced with 200 μl of CM3 or CM37. The plate was incubated at 37° C. for 15 minutes and subsequently placed at 3° C. or 37° C. for 24 hours.

To investigate the potential of serotonin in upregulating CBS expression in cells, SMAC cells were cultured in a 6 well plate one day before serotonin treatment. After reaching confluence, cells were incubated with 1.3 μM serotonin in SMAC cell complete medium. Control wells only contained the medium. At every time point (5, 10, min), cells were washed with PBS, lysed using RIPA buffer and western blotted to study the change in CBS expression level.

1.3 Western Blot, Histology and siRNA for Cystathionine-β-Synthase

To have a general live stain of the cells at 37° C. and 3° C. Methylene blue was added to the medium of the cells, and photographs were made after 2 hours in both temperatures. The presence of 5-HT was investigated by staining using two methods: Ehrlich reagent and immunostaining. To assess the presence of CBS protein inside the cells and lung tissue sections obtained from different phases of hibernation in hamster, an antibody against this protein was incorporated.

Ehrlich reagent was used after fixation with acetone (100%) for 10 min. Ehrlich's reagent was prepared by dissolving 100 mg p-dimethylaminobenzaldehyde in 100 ml 17:3 (v/v) glacial acetic acid/hydrochloric acid mixture and stored at 4° C. until later use. Fixated cells were placed inside a glass chamber containing 2% Ehrlich reagent and heated at 60° C. for 30 min. Next, slides were washed with PBS and examined using a light microscope. For immunohistological examination, cells were fixed by acetone (100%) for 10 min, washed and rehydrated with PBS. Hydrogen peroxidase activity was blocked by hydrogen peroxide (1%) in PBS, washed with PBS three times, each time for 5 min and incubated for 1 h with 1% primary antibody; Rb PAb to serotonin 50 μl (ab8882-50, Abcam) in PBS containing 1% BSA for an hour, washed in PBS trice and incubated with 1% secondary antibody (Dako po448) polyclonal Goat AntiRabbit HRP in PBS containing 1% BSA for 1 hour and again washed in PBS trice. The signal was amplified by a 1% of the third antibody (Dako po449) polyclonal Rabbit Anti Goat.

For immunohistological examination of CBS protein the same procedure as outlined above was followed to prepare the cells. Fixed cells were incubated with anti-Goat CBS antibody (Santa Cruz; CBS goat polyclonal IgG; sc-46830, USA), in PBS containing 1% BSA. The slides were washed with PBS and incubated with the second antibody; 1% Rabbit Anti-Goat/HRP (PO449, Dako, UK), in PBS containing 1% BSA, and 1% Hamster serum for 1 h and washed with PBS. To amplify the antibody signal a third antibody; 1% Goat Anti-Rabbit/HRP (PO448, Dako, UK) was applied in PBS containing 1% BSA, and 1% Hamster serum for 1 h and washed with PBS.

To investigate the role of Cystathione beta-synthase activity in the protective effect of serotonin the expression of CBS was reduced by applying a predesigned siRNA (sc-60336, Santa Cruz, USA) and compared to a silencer negative control (Ambion, AM4644, Huntingdon, UK). DDT-1 and SMAC cells at 60-80% confluence were seeded in 96 or 6 well plates in antibiotic-free normal growth medium supplemented with FCS. Cells were transfected using lipofectamine 2000 (11668-500, Invitrogen, UK) according to the protocol provided by the manufacturer (www.invitrogen.com) at a final concentration of 100 pmol siRNA in 5 μl lipofectamine for each well in a 6 well plate and 5 pmol siRNA in 0.25 μl lipofectamine for each well in a 96 well plate. After 24 h, the medium was changed to the medium containing antibiotics and FCS. Control cells, siRNA treated cells and cells with negative control silencer were incubated at 37° C. or 3° C. in the presence and absence of serotonin for 24 h, washed with PBS and lyzed in 120 μl RIPA buffer. Control cells were incubated with creatine sulfate to exclude any effect of this substance. The protein concentration was measured by Bradford assay in all the samples. Loading buffer (20 μl) was added to every 50 μg of cell protein and ran at 100V for 70 min. Proteins were transferred to a nitrocellulose membrane and detected by West Pico Chemiluminescent Substrate (supersignal), photographed and analyzed with genetool software (version 3.08, SynGene, England). The western blot results for CBS protein expression were corrected over GAPDH internal reference expression.

To analyze the expression of CBS in lungs of a hibernating animal, tissue was lyzed in RIPA buffer with the use of a homogenizer. The protein concentration in the sample tissues were measured according to Bradford protein assay. 50 μg of lung samples mixed with loading buffer were boiled and loaded into western blot 4-20% precise protein loading gel (Thermo-scientific) wells. The proteins were transferred onto a nitro-cellulose membrane and probed by CBS antibody and second antibody used for cell staining. The membranes were developed using supersignal West Dura substrate and syngene version 6.07 was used to capture the illuminated bands representing the level of protein expression. The results were analyzed using genetools version 3.08. The band intensities obtained from CBS protein were corrected over GAPDH as an internal reference. Hamster lung tissue samples from different phases of hibernation were harvested and embedded in paraffin. Paraffin blocks were cut in 3 μm sections, deparaffinized, and submitted to CBS antibody staining according to the procedure described above.

### 1.4 Quantitative Assessment of Serotonine in Cells and SERT Blockage

Ehrlich's reagent was used to quantify the cellular amount of indoles. Qualitative analysis of cellular indoles in cell culture medium at 37° C. and 3° C. was conducted after extraction according to Happold and Hoyle. Five ml of medium was shaken vigorously with 2 ml of xylene. Next, 1 ml of Ehrlich's reagent is applied to the surface of the mixture. Redistribution of xylene through the Ehrlich's reagent induces formation of the rosindole body, a red ring appearing at the lower surface of the xylene layer indicating the presence of indoleamides. The change in indole concentration in DDT-1 cells was measured after washing the cells with PBS, centrifugation (1000 rpm, 5 min) and removal of supernatant. Ehrlich reagent (200 μl) was added to each tube. After 3 min of vortexing, tubes were left for 3 h at 60° C. After centrifugation (1000 rpm, 5 min), color intensity was spectrophotometrically measured at 625 nm. Calibration experiments were carried out using 5-HT (0.025-0.5 mM), which rendered a linear regression with a correlation coefficient (R2) of 0.9996 (data not shown). To verify the accuracy of the Ehrlich reagent experiments automated mass spectrometric analysis was performed on all the samples according to the method set up by Ido P. Kema.

To assess the role of Serotonin transporter in cell survival at 3° C. confluent DDT-1 and SMAC cells were treated with a selective SERT inhibitor; Flouxetine (0.001-1 μM) for 10 min at 37° C. and later incubated with a combination of serotonin (13 nmol) and 2 concentrations of Fluoxetine for 15 min at 37° C. and placed at 3° C. for 24 hr. MTS assay was performed to investigate cell survival after blocking serotonin transporter and hypothermic treatment.

### 1.5 Production of H₂S

Methylene blue method for H₂S detection was applied to quantitatively measure the H₂S production. Cells were washed with PBS, scraped and centrifuged for 60 sec at 1000 rpm. After removal of the supernatant, zinc acetate 1% in water (200 μl) was added to the cell sediments and the cells were disrupted by small glass beads and vortexed for 20 seconds. Diamine-ferric solution was prepared by mixing 100 μl of a 400 mg N,N-dimethyl-p-phenylenediamine dihydrochloride dissolved in 10 ml 6M HCl and 100 μl of 600 mg ferric chloride in 10 ml 6M HCl. Two hundred μl of this mixture was added to the cell suspension and after an incubation time of 30 min at 37° C. and centrifugation, the amount of methylene blue formed in the supernatant was measured at a wavelength of 670 nm. To measure H₂S content of supernatant, the same procedure was repeated for the cell free medium of cells incubated at both 37° C. and 3° C. Blanks were made following the same procedure without cells or using fresh medium. The concentration of H₂S was calculated by extrapolation using a standard curve obtained from different concentrations of Methylene blue and spectrophotometric measurement at a wavelength of 670 nm. The amount of H₂S present was calculated on the basis that every mole of methylene blue formed in this reaction contains 32 g (1 mole) of captured sulfur.

### 1.6 CBS Enzyme Kinetics and Docking Analysis of Compound Binding to CBS

To examine the potential of serotonin to act as a cofactor or allosteric activator of CBS, the enzyme was isolated from DDT-1 cells. In brief, DDT-1 cells were lyzed by a non-denaturing buffer. CBS antibody (1 μg/ml) was diluted in coating solution and 100 μl of it was added to each well of a microplate. The plates were left at 4° C. over night. The wells were washed with PBS three times for 2 min and 10 μg/ml of the protein was added to each well and the microplate was left at 4° C. for another 24 hr and later washed three times with PBS. The substrates cysteine and homocysteine at the concentration of 10 μmol each were mixed and 100 μl was added to each well in the absence or presence of PLP (pyridoxal-5-phosphate) or serotonin (30 nmol) in PBS.

To investigate the possibility of serotonin fitting into the enzymatic pocket, we performed docking analysis employing a molecular docking program by Bikadi et al.

### 1.7 Inhibition of Serotonin Synthesis

Parachlorophenyl-alanine (PCPA; Sigma, C6506-5G) was dissolved in warmed, acidified (pH 6.8) DDT-1 medium and vortexed for 5 min to a final concentration of 1.25 μM. Other concentrations were made from this stock solution. Control experiments were performed with a similar solution without PCPA. Anhydrous creatine (Sigma C4255-25G, USA) was dissolved in cell medium and added to wells to exclude the effect of this component. The treatment continued for 4 days until the concentration of indoleamines inside the cells reached half the baseline value. The cells were placed at 3° C., and MTS assay was performed after 24 h.

### 1.8 Concentration of Serotonin Derivatives in Hamster Serum

Hibernation in Syrian golden hamsters (Mesocricetus auratus, n=24) was induced by lowering the ambient temperature during 3 weeks under short-day conditions from 20° C. to 5° C. and light/dark-pattern was changed to continuous dim light (<1 Lux). To assess the individual torpor or euthermic states, activity was measured every minute using a computer based recording system. Hamsters were sacrificed during subsequent phases of hibernation, i.e. early torpor (TE, 24 h at body temperature <8° C., n=4), deep/late torpor (TL, 5 days at body temperature <8° C., n=4), early arousal (EA, 1.5 hours after onset of arousal, n=4), late arousal (LA, 8 hours after reaching euthermia, n=4). Summer euthermic (EU, n=4) served as controls. The experiments were approved by the Animal Experiments Committee of the University of Groningen (DEC#4746).

Twenty μl of plasma obtained from each animal was used to measure indolamine concentration according to Narasimhachari et al. Ethyl acetate (300 μl) was added to each sample, vortexed for 10 s and centrifuged for 5 min at 2500 rpm. The ethyl acetate layer was transferred to another tube and its content was dried by cold air. Ehrlich reagent (50 μl) was added to each tube and warmed to 60° C. After 2 hr the amount of blue color representing the presence of indolamines was measured using a 384 well plate and a plate reader at 625 nm.

### 1.9 Mass Spectrometry for Serotonin

SMAC were grown to confluence in 25 T flasks. Control cells at 37° C. were incubated in PBS in the absence of presence of Fluoxetine for 15 min. Then they were incubated at either 37 or 3° C. for 24 hr. The supernatant was filtered to prepare the samples for Mass spectrophotometrical analysis of the content of serotonin. Working solutions of serotonin were diluted from a freshly weighed stock solution (1 mg/mL) on the day of analysis. Aqueous calibrators were prepared by addition of working solution corresponding to concentrations from 30 to 7,300 nmol/Lserotonin. 100 μl was injected into the XLC-MS/MS system. The mass spectrometer was directly coupled to the chromatographic column (Atlantis HILIC Silica column (particle size 3 μm, 2.1 mm internal diameter by 50 mm; Waters). In positive electrospray ionization mode serotonin and its deuterated internal standard were protonated to produce ions at the form [M+H]+, with m/z 177 and m/z 181, respectively. Upon collision-induced dissociation (CID) with argon gas, these precursor ions produced characteristic product ions of m/z 160 [M−NH2] and 132 [M−C2H4NH2] and 115 [M−C2H4NH2OH] for serotonin and m/z 164, 136, and 119 for the deuterated internal standard.

### 1.10 Statistics

Statistical data analyses were performed using the One-way ANOVA (P<0.05) with tukey test (GraphPad Prism version 5.00 for Windows, GraphPad Software, San Diego Calif. USA, www.graphpad.com), unless indicated otherwise.

### Example 1

### Hypothermia Resistance of 5 Cell Lines

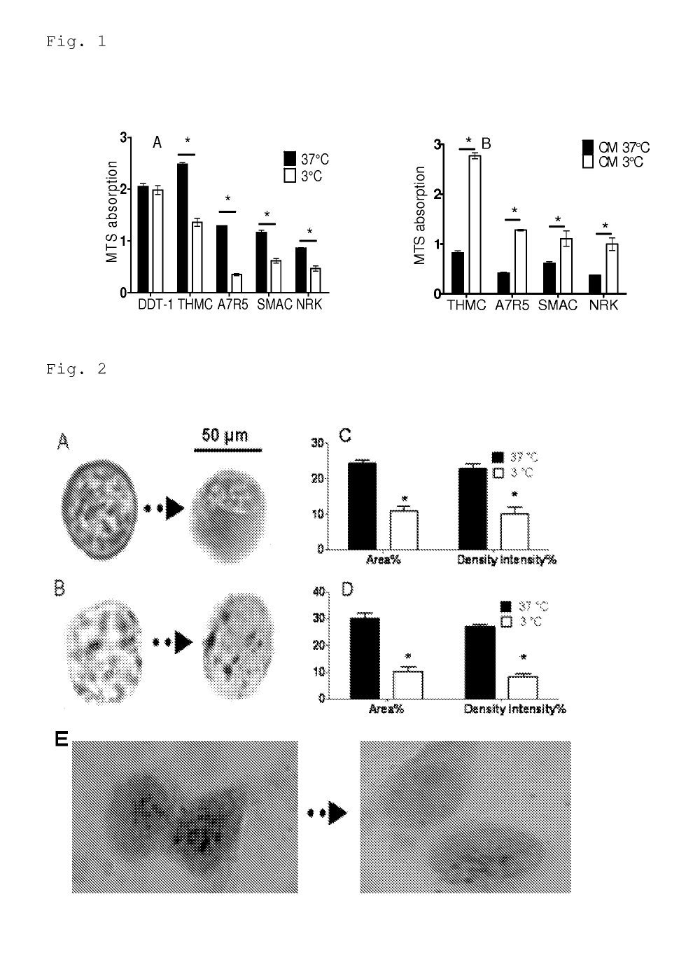

A7R5, DDT-1, NRK, SMAC and THMC cell lines were used to investigate their resistance to hypothermic injury after growing to confluence and subsequently placing at 3° C. for 24 h. During a rewarming phase of 3 h, the viability of the cells was assessed by MTS assay. Whereas DDT-1 cells fully survived the hypothermic conditions, viability of all the other cell lines was significantly decreased after 24 hr at 3° C. (FIG. 1A), demonstrating the potential of DDT-1 cells to resist hypothermic injury compared to other cell lines.

### Example 2

### Protection of Cell Lines by Medium of Hypothermic DDT-1 Cells

The protective nature of medium conditioned by hypothermic DDT-1 cells (3° C., 18 hrs; CM3) against hypothermic injury of vulnerable cell lines was investigated by comparing the effect of CM3 to medium from normothermic DDT1 cells (CM37). Cells treated with CM3 showed a significant increase in cell survival of all cell lines compared to cells treated with CM37 (FIG. 1B). Thus, hypothermia seems to be an essential factor in the process leading to the release of a protective factor from DDT-1 into the medium.

### Example 3

### Identification of Serotonin in DDT-1 Cells

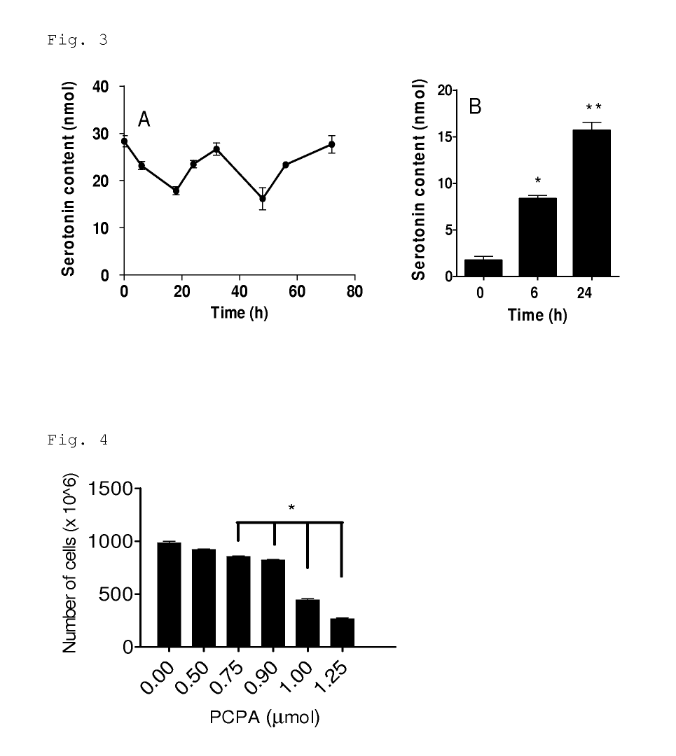

To obtain insight into possible protective factors, normothermic and hypothermic DDT1 cells were fixed and stained. Methylene blue staining performed on normothermic and hypothermic DDT-1 cells clearly displayed cytoplasmic vesicles (FIG. 2A-C). Whereas DDT-1 cells displayed a uniform distribution of staining during normothermia. A polarization of cytoplasmic content was observed in the figures following hypothermia treatment. Because of their morphology, it was hypothesized that DDT-1 vesicles may represent neurosecretory-like vesicles filled with serotonin, which are released during hypothermia. The presence of serotonin inside the vesicles was investigating by staining with Ehrlich reagent to detect specific indoleamines in hypothermic and normothermic DDT-1 cells. Whereas normothermic cells showed abundant presence of these vesicles, the staining area and intensity was significantly decreased in hypothermic DDT-1 cells (FIG. 2B-D).

The concentration of indoleamines inside DDT-1 cells was measured at various time points after induction of hypothermia in homogenized cells using Ehrlich reagent (FIG. 3A). While in normothermic cells alkaloid concentration was calculated at 30±5 nmol per 10̂6 cells, a significant decrease to about half of this value was found in hypothermic cells. Next, the concentration of indoleamines was measured in hypothermic SMAC cells treated with CM3 and CM37 to investigate the entrance of this substance into these cells. In CM37 treated SMAC cells, indoleamine concentration was similar to untreated cells (data not shown). In contrast, SMAC cells treated with CM3 displayed a 3.5 fold increase in indoleamine content already present 6 h after induction of hypothermia, which increased even further after 24 h of incubation with CM3 (FIG. 3 B). By subtracting the level inside the DDT-1 cells after 24 hr in hypothermia from the level found in the cells at 37° C., it was calculated that CM3 of 10̂6 cells contained 20 nmol serotonin. The mass spectrophotometric data confirmed the data obtained from Ehrlich reaction (table 1). The indoleamine content of the cells at 3° C. was measured during 72 h and showed a fluctuating pattern suggesting alternating secretion and reabsorption of indoleamines by these cells. The CM3 medium was obtained when the cells had the lowest content of this substance (i.e. at 18 h.)

TABLE 1
Serotonin concentration in SMAC cells established
by Mass Spectometry.

| | Conc. Serotonine |
|---|---|
| condition: | (nmol/l): |
| PBS-blank | <3.0 |
| PBS 3° C. | 25.0 |
| PBS 37° C. | <3.0 |
| Fluoxetine | 20.5 |
| 37° C. | |
| Fluoxetine 3° C. | 20.1 |

Cells were pretreated for 15 min and incubated at given temperatures for 24 h.

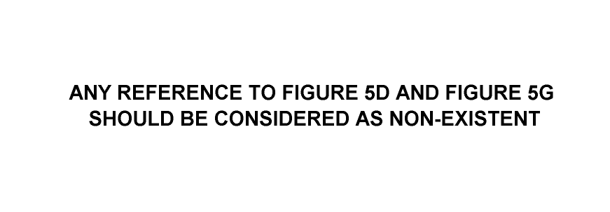

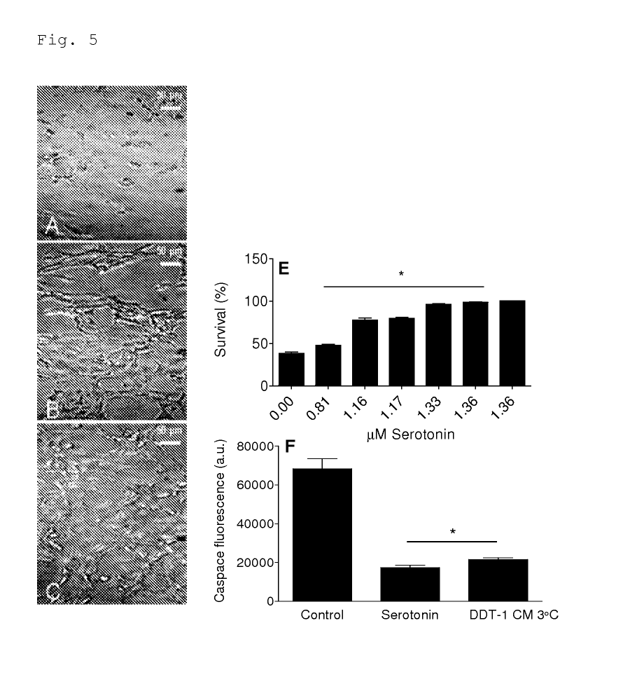

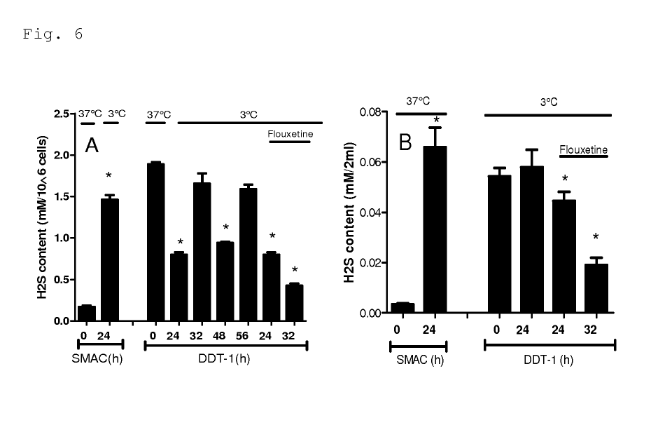

To further identify the indoleamine involved, an inhibitor of tryptophan hydroxylase parachlorophenylalanine (PCPA) was added to the medium to block synthesis of serotonin. A decrease of 50±10% in indoleamine content of the cells was noted after 4 days of pretreatment of the cells by PCPA (n=8). Pretreatment of normothermic DDT-1 cells with PCPA started to show the decrease in cell indolamine level after 48 h. It was noted the PCPA concentration-dependently decreased DDT-1 survival following a subsequent period of hypothermia (48 hr, 3° C.; FIG. 4). To further substantiate involvement of serotonin, its protective action on hypothermic cell death was investigated by adding serotonin (5 nmol/L) to SMAC cells 15 min before the initiation of hypothermia. The substantial reduction in number of cells observed in untreated cells was concentration-dependently prevented by serotonin to a similar extend as found by CM37 (FIG. 5 A-E). In addition, marked apoptosis was observed in hypothermic SMAC cells, which was completely and dose dependently attenuated both by CM37 and serotonin (5 nmol/L; FIG. 5 F). Creatine sulfate did not show any protective effects on cells (data not shown).

To investigate involvement of 5-HT2 receptors, the experiment was repeated in the presence of ketanserine. Ketanserine (400 ng/ml and 10 μg/ml) did not affect the resistance of DDT-1 cells to hypothermia (24 h, 3° C.), nor did it affect the protective effect of serotonin on hypothermic SMAC cells (data not shown). To investigate whether the uptake of serotonin via its transporter (SERT) was implicated in its protective effect, cells were incubated with fluoxetine (0.005 μM). Blockade of SERT on DDT-1 cells with flouxetine 15 minutes before hypothermic treatment (24 h) resulted in death of more than half of these cells (FIG. 5G). Similarly, blockade of SERT resulted in the complete annihilation of the protective effect of serotonin against hypothermic cell death in confluent SMAC cells (FIG. 5G).

Together, these experiments demonstrate that the protective effect of serotonin is dependent on its uptake via SERT and exclude the involvement of 5-HT2 receptors.

### Example 4

### Protection by Serotonin Involves H₂S

It was noted that medium from hypothermic DDT-1 cells slightly smelt of rotten eggs indicating a potential production of H₂S in these cells. Therefore, H₂S content was measured in homogenates of DDT-1 and SMAC cells by Methylene blue method. H₂S content in untreated DDT-1 cells (24 h, 37° C.), amounting 1.8±0.5 mmol per 10̂6 cells, decreased about 3 fold during hypothermia (24 h, 3° C.), to 0.5±0.9 mmol per 10̂6 cells. This low level increased again after 32 h, decreasing 48 and increasing again at 56 h demonstrating a pattern of fluctuation similar to those found for serotonin content of these cells at 3° C. (FIG. 6A). In hypothermic SMAC cells (24 h, 3° C.), serotonin pretreatment (15 min, 1.3 μM), increased H₂S content 8 fold by from 0.17±0.04 to 1.4±0.2 μmoles per 10̂6 cells in untreated and serotonin treated cells, respectively. The concentration of H₂S in the medium of DDT-1 cells was 55±4 μM at 37° C. In contrast, the very low level of H₂S in medium of SMAC at 37° C. (1.5 μM) increased 20 times at 3° C. reaching the level of H₂S found in DDT-1 medium (FIG. 6). Fluoxetine treated DDT-1 cells show a lower concentration of H₂S inside the cells at 24 h that decreases even more after 32 h, but the H₂S inside the medium stays constant getting lower only after 32 h of hypothermic treatment. No fluctuation in H₂S concentration of SMAC cells was observed during 56 h (16, 24, 32, 56 h) (data not shown.)

### Example 5

### Cystathionine-β-Synthase Mediates Protection by Serotonin

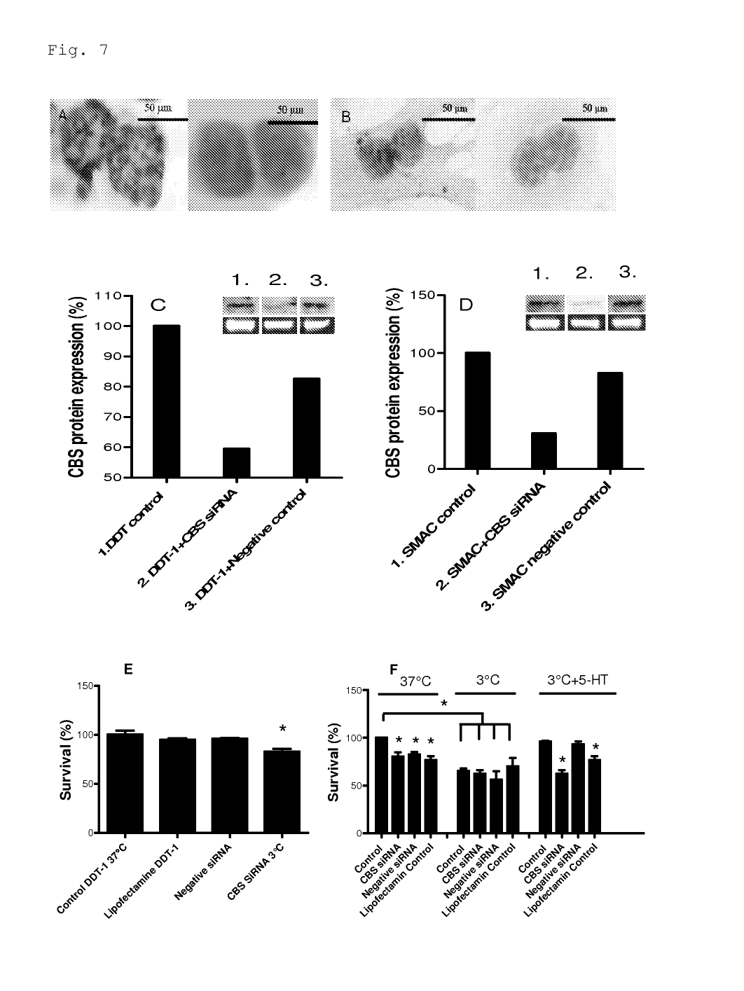

Cystathionine-β-synthase (CBS) is one of the main enzymes implicated in the production of H₂S. Both DDT-1 and SMAC cells were fixed by acetone, stained using CBS antibody and compared to controls. Histological examination confirmed the presence of the enzyme both in DDT-1 and SMAC cells (FIG. 7 A,B). To confirm that the protective effect of serotonin is due to CBS mediated production of H₂S, expression of the enzyme was reduced using siRNA both in DDT-1 and SMAC cells. CBS siRNA substantially reduced CBS expression of both DDT-1 and SMAC cell lines compared to control (FIG. 7 C,D). Reduction of CBS expression decreased the survival of DDT-1 cells in hypothermic conditions (FIG. 7E). Also, CBS siRNA treatment annihilated the protective effect of serotonin on hypothermic cell death in SMAC cells (FIG. 7F). Thus, knockdown of CBS using siRNA implicate CBS to be involved in the resistance of DDT-1 to hypothermic conditions and demonstrates that the protective effect of serotonin on SMAC is mediated via CBS.

### Example 6

### Serotonin Upregulates CBS in SMAC Cells

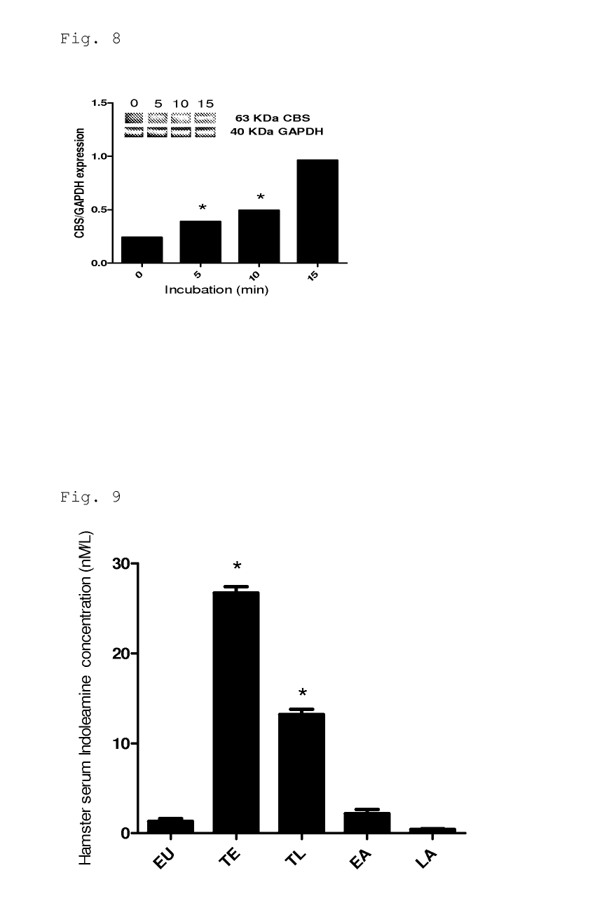

As serotonin was administered to SMAC cells 15 min prior to hypothermic treatment, its effect on expression of CBS was measured at incubation at 37° C. During the 15 min time interval, CBS expression was induced 4-fold by pretreatment with serotonin (FIG. 8)

### Example 7

### Concentration of Serotonin Derivatives in the Serum Obtained from Hamsters

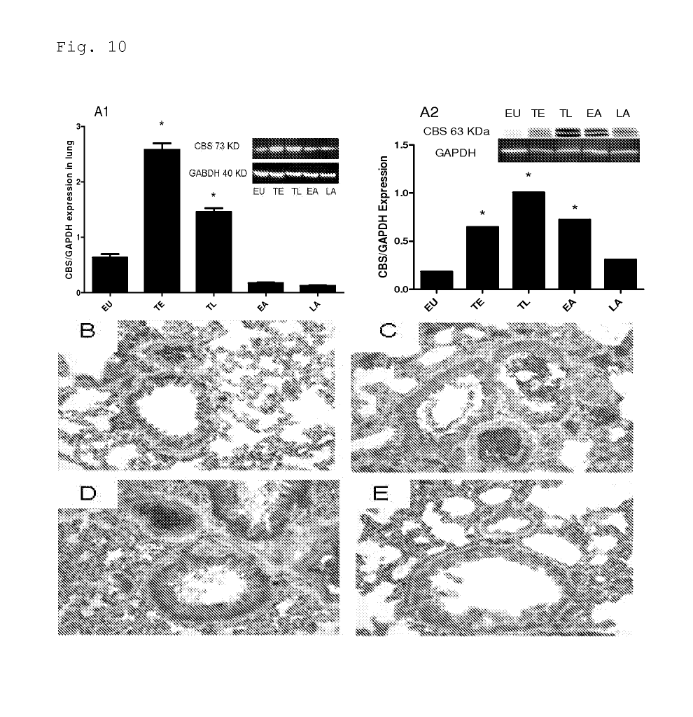

To investigate whether the concentration of serotonin also changes during different phases of hibernation in hamster, the serum serotonin concentration was measured. The data demonstrate a significant rise of 7.5-fold increase during TE that decreases in TL and returns to baseline levels in arousal (FIG. 9).

### Example 8

### CBS Protein Staining of Hamster Lung Tissue

Finally, to investigate whether CBS is implicated in the protection of cells against hypothermic damage under physiological conditions, its expression was measured in lungs of hibernating animals during phases with low body temperature (torpor (TE,TL): 7.9±0.4° C., n=8) and normal body temperature (arousal (EU, EA, LA): 36.6° C.±0.3° C., n=12).

Western blot showed a 3-fold upregulation of CBS expression during the early phase of torpor compared to summer euthermic animals, which decreased to a 2-fold upregulation at the end of the torpor bout (FIG. 10A). Importantly, expression of CBS was normalized both after short and long-term arousal (FIG. 10A1). Immunohistology was performed to investigate localization of CBS in hibernating animals. In summer euthermic and aroused animals expression was confined to few of the cells surrounding the bronchioles and alveoli (FIG. 10B,C). During torpor, expression was increased mainly in TE compared to TL. Whole blood pellets obtained from animals in each state was also examined for the increase in expression of this protein. CBS expression was increased during TE and increased further during TL. During EA it decreased, reaching a normal level at LA. Thus, the increase in CBS expression in blood cells lags behind that found in tissue CBS, (FIG. 10A2).

### Example 9

### Docking Analysis of Serotonin Binding to CBS and H₂S Production by Isolated CBS Enzyme

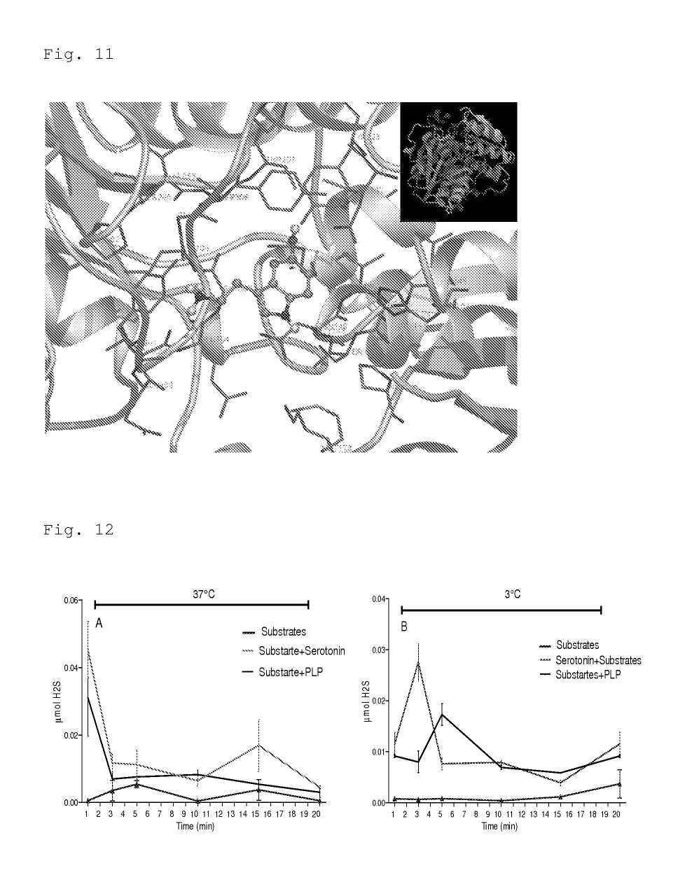

In addition to upregulating CBS, serotonin may activate the protein through allosteric binding. Previous studies demonstrated various compounds, including Pyridoxal 5-Phosphate (PLP) and S-Adenosyl methionine (SAM), to bind to the CBS domain of the protein and activate CBS leading to the production of H₂S. The structure of serotonin shows clear similarity to PLP and SAM. Modeling studies showed serotonin to bind to the same pocket as PLP to form a stable binding with a free energy of binding of −4.8 Kcal/mol, which is similar to that reported for PLP (−4.81 kcal/mol). By comparing PLP and serotonin according to the inhibition constant, the electrostatic energy, dissolve energy and the total internal energy it's clear that these properties are not significantly different (FIG. 11). Hydrogen bindings, Polar interactions, pi-pi interactions, hydrophobic interactions, cation-pi interactions and other protein-ligand interactions stabilize this binding further (data not included). Together, these data implicate that serotonin binds to CBS in a manner similar to PLP hence we hypothesis that it could activate the enzyme in a manner other than increasing the expression of CBS but also by activating the protein itself to produce H₂S.

| Substarter/ligand type | Serotonin |
|---|---|
| Est. Free energy of binding (Kcal/mol) | −4.84 |
| Est. Inhibition constant, Ki (uM) | 282.37 |
| vdW + Hbond + desolv. Energy (Kcal/mol) | −5.47 |
| Electrostatic Energy (Kcal/mol) | −0.52 |
| Total Internal Energy (Kcal/mol) | −6.1 |

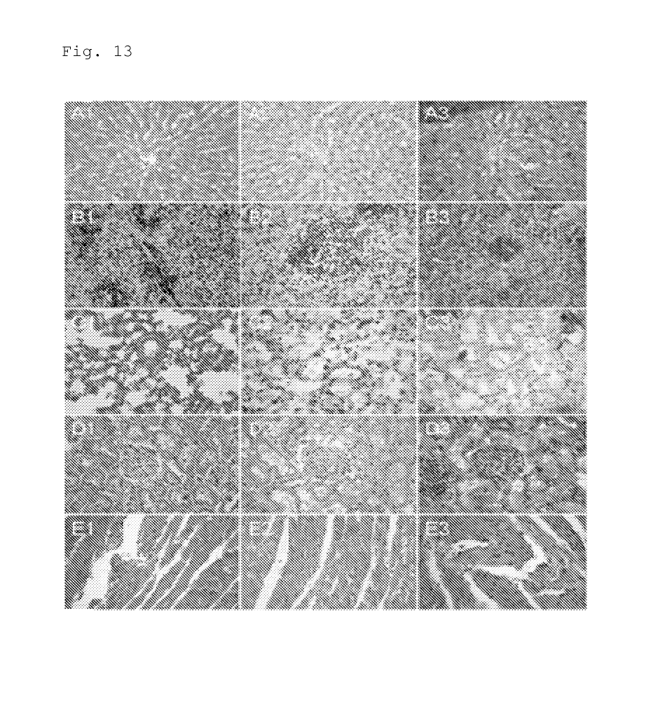

Finally, serotonin was found to increase the activity of isolated CBS, both at 37° C. and 3° C. (FIG. 12).

### Example 10

### CBS Expression in Normothermic and Hypothermic Tissue

Two rats (rattus norvegicus) were sacrificed and blood was taken out. The tissues were flushed by either PBS as control or PBS plus dopamine. Liver, pancreas, lung, kidney and heart were harvested. Tissue samples were harvested and kept at room temperature for 15 min and then divided among 3 groups: control at 37° C., control in PBS at 3° C. and protective factor-PBS at 3° C. for 24 hr. Tissues were fixed after being taken out of cold room. The tissues were later embedded in Paraffin, cut into 5 μm sections and stained with CBS antibody.

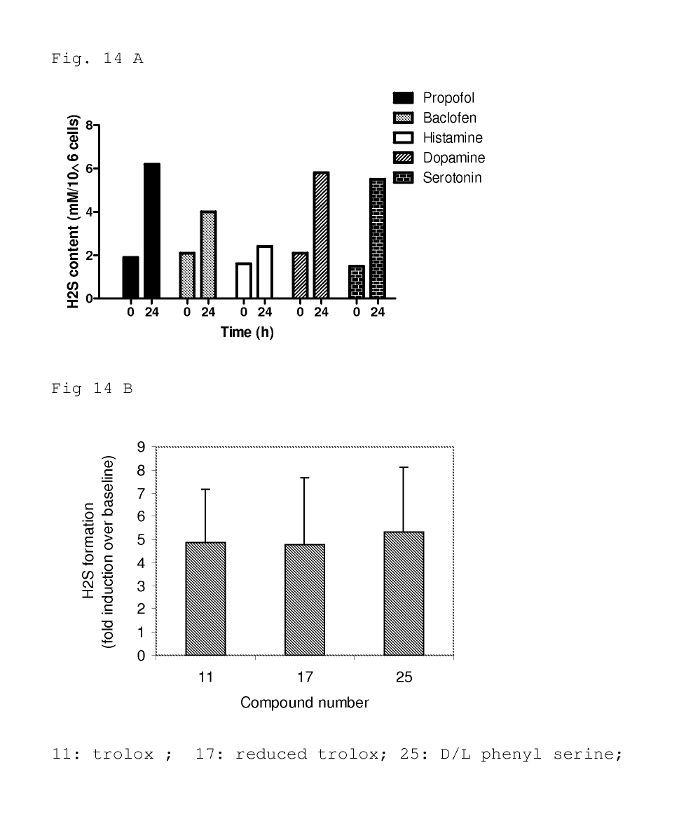

Results show a downregulation of CBS following hypothermia, but an upregulation of CBS in the presence of dopamine (FIG. 13). Similar results were obtained for serotonin.

### Example 11

### H₂S Production by Propofol, Baclofen, Histamine, Dopamine, Melatinon, D/L Phenylserine, Trolox, Reduced Trolox and Serotonin

Methylene blue method for H₂S detection was applied to quantitatively examine the H₂S present in cell supernatant at 0 and 24 hr after hypothermic treatment at 3° C.

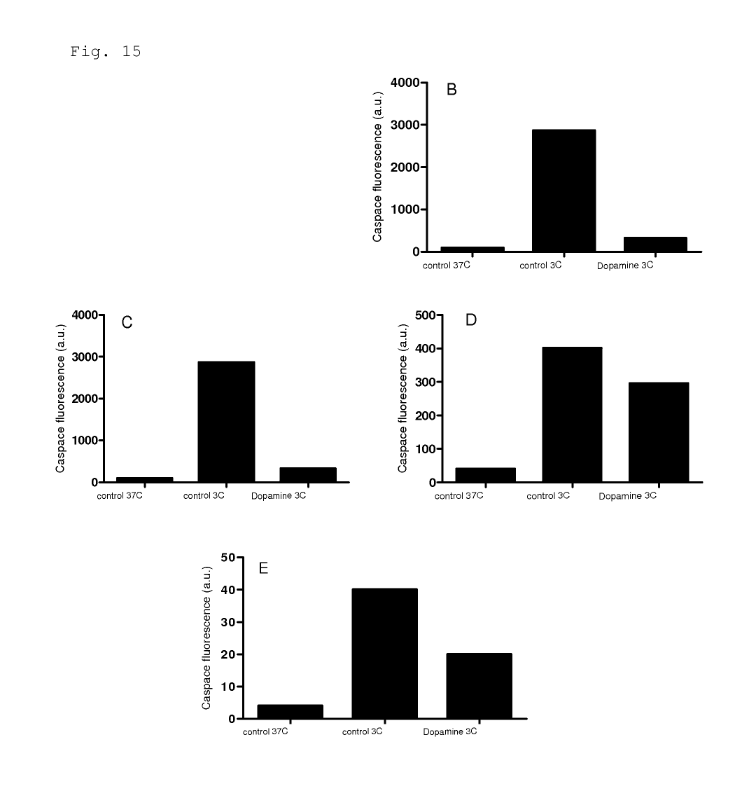

SMAC cells incubated at 3° C. for 24 hour show increased H₂S production following incubation with propofol, baclofen, histamine, dopamine, melatonin (FIG. 14 A). The same test was performed with trolox, reduced trolox, and D/L phenylserine (FIG. 14 B).

### Example 12

### Dopamine Prevents Apoptosis in Hypothermic Tissue

Tissue samples were harvested and kept at room temperature for 15 min and then divided among 3 groups: control at 37° C., control in PBS at 3° C. and protective factor-PBS at 3° C. for 24 hr. The tissues were lyzed with RIPA buffer and the protein concentration in each sample was calculated using the Bradford assay. Caspace 3/7 assay was conducted on 50 μg protein from each sample to study the apoptosis in each tissue.

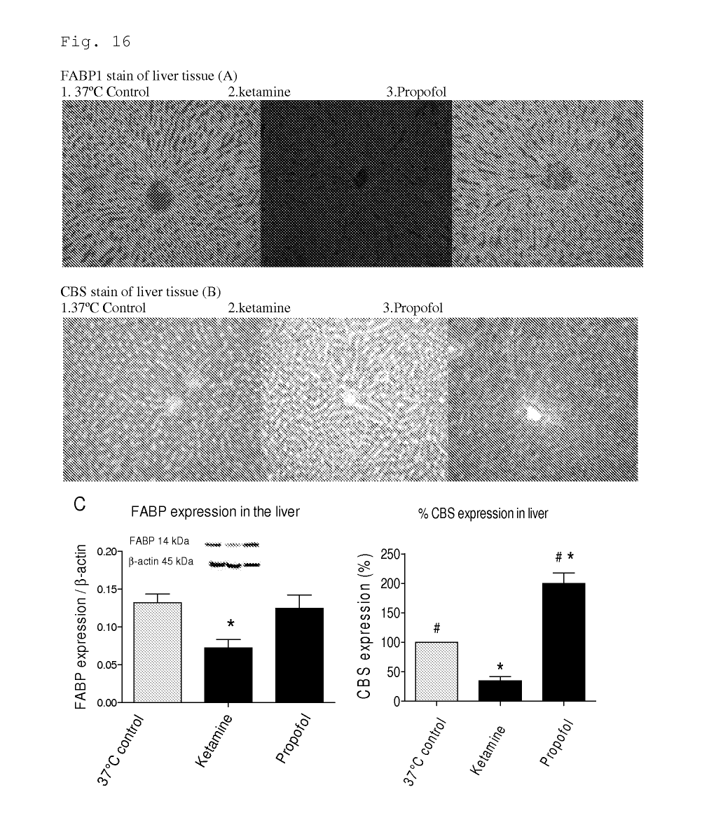

Results show a reduction of caspase activity in tissue stored under hypothermia, which is abrogated by incubation with dopamine (FIG. 15).

### Example 13

### In Vivo Protection from Hypothermic Damage in Rats

To investigate the protective effects of the proposed mechanism from hypothermic damage, rats were cooled down to 15° C. body temperature for 3 hr and re-warmed to 37° C. for 1 hr. Organ damage was assessed in liver.

### Animals

Three groups of male Wistar rats (350-400 gr) were investigated: cooled rats either anesthetized with propofol or ketamine (n=6 each); a non-cooled control group briefly anesthetized with isofluran was sacrificed at 37° C. (n=3). After anesthesia with isofluran, the trachea was intubated with a 6.0-mm cuffed tube and mechanical ventilation was started with air. A 5F central venous catheter was introduced in the internal jugular vein for blood sampling, hydration and administration of the anesthetic. A catheter was inserted in the common carotid artery for continuous monitoring of systemic arterial pressure and for blood gas analysis. Cardiovascular and oxygen saturation monitoring was performed during the entire procedure. After preparation, isoflurane was stopped and anesthesia was maintained with either propofol-Lipro (20 mg/m; Braun, Melsungen, Duitsland) at 2 ml/h or ketamine 0.6 ml/hr for 1 hr. Cooling of the animals was accomplished by an external cooling blanket and ice packs. A gradual cooling was achieved at a rate of 1° C. every 3 min to 15±0.5° C. while lowering the anesthetic infusion rate to 0.2 ml/hr for both propofol and ketamine. Rats were kept at this temperature for 3 hr, followed by rewarming to 37° C. in about 1 hr using a heating blanket and warm air. The rats were then kept at 37° C. for another hr, followed by sacrification by exsanguination. Blood sampling was performed at the end of the preparation period and every hr after the body temperature reached 15° C. Liver biopsies were obtained and immediately fixated in paraformaldehyde (4%) or snap frozen in liquid nitrogen. The family of Fatty Acid Binding Proteins (FABP) is tissue specific and is used as a damage marker. Ischemically damaged tissues release FABP rapidly enabling early detection of organ damage.

### Protein Measurement in Liver

To assess organ damage, the expression of FABP1 (Santa Cruz, L-FABP/FABP1 SC50380), an early liver damage marker, was studied in 5 μm slices from paraffin blocks by immunostaining. The expression of FABP1 was also measured using western blotting.

CBS (cystathionine betasynthase) expression was also measured as the one of the main enzymes producing H₂S in the liver.

### Serum H₂S Concentration

Methylene blue method for H₂S detection was applied to quantitatively measure the H₂S content in serum obtained from non treated controls, propofol and ketamine rats. The diluted serum samples obtained from whole blood (25 μL in 50 mmol/L potassium phosphate buffer, pH 8.0) were mixed with 0.25 mL Zn acetate (1%) and 0.45 were mixed with N,N-dimethyl-p-phenylenediamine sulfate (20 mmol/L; 133 μL) in 7.2 mol/L HCl and FeCl3 (30 mmol/L; 133 μL) in 1.2 mol/L HCl. After 20 minutes, absorbance was measured at 670 nm. Blanks were made following the same procedure without samples. The concentration of H₂S was calculated by extrapolation using a standard curve obtained from different concentrations of methylene blue and spectrophotometric measurement at a wavelength of 670 nm (Tripatara et al., 2009; Uchida et al., 2000).

### Results

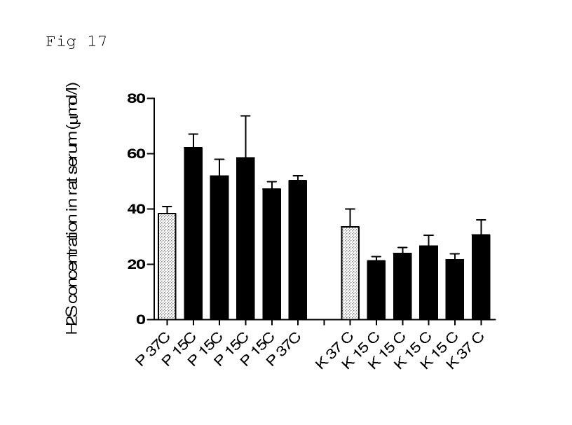

Cooled animals anesthetized with ketamine show substantial liver damage, as demonstrated by the decreased expression of FABP1 (FIG. 16 panels A2,C) compared to control. Rats anesthetized with propofol are protected from hypothermia induced liver damage, as FABP1 expression is similar to control non-cooled animals (FIG. 16 panels A,C). Propofol anesthesia increased the expression of CBS in liver compared to controls, while in ketamine anesthetized animals CBS levels were significantly lowered (FIG. 16 panels B,D). Serum H₂S concentration was increased after administration of propofol, while a decreased concentration of serum H₂S was found upon anesthesia with ketamine (FIG. 17).

Administration of propofol upregulates CBS expression in liver of hypothermic rats, and protects the organ against hypothermic damage. This is accompanied by increased serum levels of H₂S.

These experiments thus provide evidence for the application of the identified mechanism in in vivo animals.

In the study the inventors demonstrate that hamster DDT-1 cells are protected from hypothermic injury due to the existence of serotonin inside these cells and the subsequent secretion of this substance into the medium leading to the protection of different cell lines vulnerable to hypothermia induced cell death. In SMAC cells, this protection was demonstrated to be due to CBS mediated production of H₂S, dependent on the uptake of serotonin via SERT and the subsequent rapid upregulation of CBS. In addition, QSAR studies show serotonin to dock at CBS at a similar pocket as known sterical activators, possibly implying induction of the enzyme's activity by serotonin. Finally, we demonstrate upregulation of CBS in lung tissue of hibernating hamster during hypothermic bouts (torpor), indicating that a subsequent increase in production of H₂S that could be a protective factor at low body temperature in hibernators. Together these data identify serotonin effects on CBS regulation as an extensive cellular protective mechanism against hypothermic cell death.

Previous data corroborate the presence of serotonin filled vesicles in vas deferens from which DDT-1 cells are derived. Fuenmayor et al. (1976a) and Celuch and Slole (1989) described the presence and release of serotonin, dopamine and noradrenalin (NA) from rat vas deferens. It is conceivable that protection from hypothermia in SMAC cells is dependent on the cellular uptake of serotonin, in view of the failure of its protection in the presence of an SSRI and the unchanged effectiveness of serotonin in the presence of the non-selective HT2 receptor blocker ketanserin. Such view is substantiated by the strongly increased cellular serotonin content of serotonin treated hypothermic SMAC cells.

These experiments demonstrate rapid upregulation of CBS as a prime mechanism of the action of serotonin. Our results with siRNA against the enzyme clearly demonstrate protection of SMAC from hypothermic cell death to be dependent on expression of CBS. CBS is a cytoplasmic and nuclear protein that operates in the first step of homocysteine transulfuration by catalyzing the formation of cystathionine from homocysteine using pyridoxal phosphate (PLP) as cofactor. Catabolism of the amino acids L-cysteine and homocysteine by CBS generates appreciable levels of H₂S. Allosteric activation by S-adenosyl-methionine (AdoMet) regulates CBS activity and PLP is a cofactor regulating the action of this protein. Transsulfuration, on the other hand, is enhanced by the stimulatory effect of AdoMet on CBS activity. In view of similarity in binding of serotonin and PLP, serotonin can also activate the enzyme. Serotonin can act as a cofactor by providing the reducing equivalents in reactions. One route for the catabolic removal of homocysteine in mammals begins with the pyridoxal phosphate-(PLP-) dependent beta-replacement reaction catalyzed by cystathionine beta-synthase. This enzyme has a b-type heme with unusual spectroscopic properties but as yet unknown function. The enzyme has a modular organization and can be cleaved into an N-terminal catalytic core, which retains both the heme and PLP-binding sites and is highly active, and a C-terminal regulatory domain, where the allosteric activator S-adenosylmethionine can bind. It can also bind a site as SAM on the enzyme.

The inventors showed that it is possible to upregulate CBS before hypothermic treatment to achieve its beneficial effects. Our data indicate that this potential of endogenously produced H₂S may be disclosed via a relatively simple pharmacological approach to enhance cell survival in medical conditions such as transplantation, ischemia/reperfusion, and hypothermia.

Finally, the inventors demonstrated that CBS is strongly induced in the hamster lung during the torpor phase of hibernation, but is rapidly normalized during arousal. This observation may signify that a H₂S mediated protective mechanism(s) are recruited during hibernation. In addition, previous studies reported that inhalation of H₂S induces a state of suspended animation in mice, characterized by decreased metabolic rate and loss of control of body temperature. Thus, upregulation of CBS may also constitute production of H₂S necessary for induction and maintenance of hibernation.

1. A compound capable of increasing or maintaining the H2S level in a cell for use in the prevention of cell injury and/or protection of a cell.

2. The compound of claim 1, wherein the increase or maintenance of the H2S level in the cell is mediated by endogenous H2S production in a cell for use in the prevention of cell injury and/or protection of a cell.

3. The compound of claim 1, wherein the compound is selected from the group consisting of serotonin, baclofen, dopamine, propofol, melatonin, histamine, D/L phenylserine, trolox, reduced trolox and/or a salt, a derivate, or a precursor thereof.

4. The compound of claim 1, wherein the endogenous H2S production is mediated by cystathionine beta synthase (CBS).

5. The compound of claim 1, wherein the compound is transferred into the cell via active transport.

6. The compound of claim 1, wherein the prevention of cell injury and/or the protection of a cell against injury is achieved in the treatment of subjects suffering from a disorder that mediates oxidative stress to cells.

7. The compound of claim 1, wherein the prevention of cell injury and/or protection of the cells is achieved in the treatment of subjects suffering from ischemic injury and/or reperfusion, neuromodulation, hypertension, inflammation, hemorrhagic shock, hypothermia, diabetes or edema.

8. The compound of claim 1, wherein the prevention of the cell injury and/or protection of the cell is achieved in conditions of therapeutic hypothermia.

9. The compound of claim 1, wherein the prevention of cell injury and/or protection of the cells is achieved in hypothermic storage of organs, tissues or cells.

10. The compound of claim 1, wherein the prevention of cell injury and/or protection of the cells is achieved during the process of rewarming organs, tissues or cells.

11. The compound of claim 1, wherein prevention of cell injury and/or protection of cell is effected by local administration via a stent or catheter.

12. A pharmaceutical composition capable of increasing or maintaining the H2S level in a cell for use in the prevention of cell injury and/or protection of a cell comprising one or more of the compounds according to claim 1 and a suitable excipient.

13. The pharmaceutical composition of claim 12, wherein prevention of cell injury and/or protection of cell is effected by oral, intravenous, subcutaneous, tracheal, bronchial, intranasal, pulmonary, transdermal, buccal, rectal, parenteral administration.

14. A composition for increasing or maintaining the H2S level wherein prevention of cell injury and/or protection of a cell is achieved, comprising one or more of the compounds selected from the group consisting of serotonin, baclofen, dopamine, propofol, melatonin, histamine, D/L phenylserine, trolox, reduced trolox and/or a salt, a derivate, or a precursor thereof and a preserving component.

15. Method for the protection of cells, cell in organs, or a tissue or preventing cell injury in cells, an organ or tissue, comprising the addition of a compound selected from the group consisting of serotonin, baclofen, dopamine, propofol, melatonin, histamine, D/L phenylserine, trolox, reduced trolox and/or a salt, a derivate, or a precursor thereof, wherein the compound or the composition is added to cells, the organ or the tissue before cooling the cells, organ or tissue.

16. Method for the protection of cells, an organ, or a tissue or preventing cell injury in cells, an organ or tissue, comprising the addition of a compound selected from the group consisting of serotonin, baclofen, dopamine, propofol, melatonin, histamine, D/L phenylserine, trolox, reduced trolox and/or a salt, a derivate, or a precursor thereof, wherein the compound or the composition is added to cells, the organ or the tissue before warming the cells, the tissue or the organ.

17. Use of a compound selected from the group consisting of serotonin, baclofen, dopamine, propofol, melatonin, histamine, D/L phenylserine, trolox, reduced trolox and/or a salt, a derivate, or a precursor thereof, for inducing suspended animation, by administering the compound to a subject.
